(19) United States
(12) Patent Application Publication
(10) Pub. No.: US 2024/0299735 A1
(43) Pub. Date: Sep. 12, 2024

(54) CONTROL CIRCUIT AND CONTROL METHOD FOR ELECTRO-STIMULATION THERAPEUTIC INSTRUMENT FOR NEUROMODULATION

(71) Applicants: Fasikl Incorporated, DALLAS, TX (US); Hangzhou Fasikl Technology Co.,Ltd, Hangzhou (CN)

(72) Inventors: Wei Wang, Hangzhou (CN); Baitong Wang, Hangzhou (CN); Jules Anh Tuan Nguyen, DALLAS, TX (US); Bing Ye, Hangzhou (CN)

(21) Appl. No.: 18/666,776

(22) Filed: May 16, 2024

(51) Int. Cl. A61N 1/02

(52) U.S. Cl. CPC A61N 1/025

(57) ABSTRACT

Disclosed are a control circuit and control method for an electro-stimulation therapeutic instrument for neuromodulation. The control circuit includes a first switch, a second switch, a third switch, a fourth switch, a first signal end, a second signal end and a constant current source, where a first end of the first switch is connected to a power supply, and a second end of the first switch is connected to the first signal end; a first end of the second switch is connected to the power supply, and a second end of the second switch is connected to the second signal end; a first end of the third switch is connected to the first signal end, and a second end of the third switch is connected to the constant current source; and a first end of the fourth switch is connected to the second signal end.

### TECHNICAL FIELD

The present disclosure relates to an electro-stimulation therapeutic instrument for neuromodulation, and in particular to a control circuit and control method for an electro-stimulation therapeutic instrument for neuromodulation.

### BACKGROUND

An H-bridge circuit and a constant current source generally serve to achieve a current constant current stimulation mode of an electro-stimulation therapeutic instrument for neuromodulation. As shown in FIG. 1, the H-bridge circuit is electrically connected to a load, and two ends of the load are further connected to electrode plates. The H-bridge circuit includes a first switch SW1, a second switch SW2, a third switch SW3, and a fourth switch SW4. When the first switch SW1 and the fourth switch SW4 are controlled to be closed through a single chip microcomputer, a forward current is output on the load; and when the second switch SW2 and the third switch SW3 are controlled to be closed through the single chip microcomputer, a reverse current is output on the load. With an incapability of the current to suddenly change, the constant current source will control its operational amplifier to overshoot when no current is output on the load. Consequently, a large current overshoot will be caused at the moment the switch is turned on. A prolonged instantaneous high current can damage the electrode plate or cause a tingling sensation to human skin, thereby affecting service life of the electrode plate and use experience by a user.

### SUMMARY

In order to overcome the defects of the prior art, a first objective of the present disclosure is to provide a control circuit for an electro-stimulation therapeutic instrument for neuromodulation, which can solve the problem that an existing electro-stimulation therapeutic instrument generates a large overshoot current at the moment of switching, thereby damaging an electrode plate or causing a tingling sensation to a human body.

A second objective of the present disclosure is to provide a control method for an electro-stimulation therapeutic instrument for neuromodulation, which can solve the problem that an existing electro-stimulation therapeutic instrument generates a large overshoot current at the moment of switching, thereby damaging an electrode plate or causing a tingling sensation to a human body.

A first objective of the present disclosure is achieved through the following technical solution:
a control circuit for an electro-stimulation therapeutic instrument for neuromodulation includes a first switch, a second switch, a third switch, a fourth switch, a constant current source, a first signal end and a second signal end, where a first end of the first switch is connected to a power supply, and a second end of the first switch is electrically connected to the first signal end; a first end of the second switch is connected to the power supply, and a second end of the second switch is electrically connected to the second signal end; a first end of the third switch is electrically connected to the first signal end, and a second end of the third switch is electrically connected to the constant current source; a first end of the fourth switch is electrically connected to the second signal end, and a second end of the fourth switch is electrically connected to the constant current source; and the first signal end is electrically connected to a first end of a load, and the second signal end is electrically connected to a second end of the load; when a forward current needs to be output to the load, the first switch is controlled to be closed, and the second switch is controlled to be closed; then the first switch is kept closed, the second switch is kept closed, and the fourth switch is controlled to be closed; and finally the fourth switch is kept closed, the first switch is kept closed, and the second switch is controlled to be disconnected, such that a current of the constant current source flows through the load to output the forward current to the load; and
when a reverse current needs to be output to the load, the first switch is controlled to be closed, and the second switch is controlled to be closed; then the first switch is kept closed, the second switch is kept closed, and the third switch is controlled to be closed; and finally, the third switch is kept closed, the second switch is kept closed, and the first switch is controlled to be disconnected, such that a current of the constant current source flows through the load to output the reverse current to the load.

Further, the control circuit further includes a main control microcontroller unit (MCU), where the MCU is electrically connected to the first switch, the second switch, the third switch, the fourth switch and the constant current source; and the main control MCU is configured to control a magnitude of a current flowing through the load from the constant current source.

Further, the first switch is a first positive channel metal oxide semiconductor (PMOS) transistor, the second switch is a second PMOS transistor, the third switch is a first negative channel metal oxide semiconductor (NMOS) transistor, and the fourth switch is a second NMOS transistor;
a source of the first PMOS transistor is connected to the power supply, a drain of the first PMOS transistor is electrically connected to the first signal end, and a gate of the first PMOS transistor is connected to a first control signal of the main control MCU; a source of the second PMOS transistor is connected to the power supply, a drain of the second PMOS transistor is electrically connected to the second signal end, and a gate of the second PMOS transistor is connected to a second control signal of the main control MCU; a source of the first NMOS transistor is electrically connected to the first signal end, a drain of the first NMOS transistor is electrically connected to the constant current source, and a gate of the first NMOS transistor is connected to a third control signal of the main control MCU; and a source of the second NMOS transistor is electrically connected to the second signal end, a drain of the second NMOS transistor is electrically connected to the constant current source, and a gate of the second NMOS transistor is connected to a fourth control signal of the main control MCU.

Further, the main control MCU is a single chip microcomputer.

Further, he first control signal, the second control signal, the third control signal and the fourth control signal are all pulse width modulation (PWM) signals; and
the main control MCU is further configured to adjust duty ratios of the corresponding PWM signals to control closing duration of the corresponding MOS transistors.

Further, the control circuit further includes a current sampling circuit, where one end of the current sampling circuit is electrically connected to the constant current source, the other end of the current sampling circuit is electrically connected to the main control MCU, and the current sampling circuit is configured to sample an actual current of the constant current source to send the actual current to the main control MCU, such that the main control MCU determines whether the electro-stimulation therapeutic instrument for neuromodulation is abnormal according to the actual current of the constant current source; and the current sampling circuit includes a sampling resistor, where the constant current source is electrically connected to the main control MCU by means of the sampling resistor.

Further, the power supply is a high-voltage power supply, and the high-voltage power supply is 50 V to 150 V.

Further, the control circuit includes a power supply module and a booster circuit, where the power supply module is electrically connected to the booster circuit; and the booster circuit is configured to boost a power supply supplied by the power supply module to obtain the power supply.

Further, the power supply module is a 3.7 V lithium battery.

A second objective of the present disclosure is achieved through the following technical solution:
a control method for an electro-stimulation therapeutic instrument for neuromodulation is applied to the control circuit for an electro-stimulation therapeutic instrument for neuromodulation used in the first objective of the present disclosure;
when a forward current needs to be output to a load, the control method includes:
step 21, controlling a first switch to be closed, and controlling a second switch to be closed;
step 22, keeping the first switch closed, keeping the second switch closed, and controlling a fourth switch to be closed; and
step 23, keeping the fourth switch closed, keeping the first switch closed, and controlling the second switch to be disconnected, so as to make a current of a constant current source flow through the load to output the forward current; and
when a reverse current needs to be output to the load, the control method includes:
step 11, controlling the first switch to be closed and the second switch to be closed;
step 12, keeping the first switch closed, keeping the second switch closed, and controlling a third switch to be closed; and
step 13, finally, keeping the third switch closed, keeping the second switch closed, and controlling the first switch to be disconnected, so as to make a current of the constant current source flow through the load to output the reverse current.

Compared with the prior art, the present disclosure has the beneficial effects:

According to the present disclosure, switching time sequences of the four switches in the control circuit for an electro-stimulation therapeutic instrument for neuromodulation are controlled, such that the current of the constant current source is output to the load after the current of the constant current source enters a stable state in advance, thereby solving the problem that due to the incapability of the current to suddenly change, a large overshoot current is generated at the moment of switching in the prior art, thereby damaging an electrode plate or causing a tingling sensation to human skin.

### BRIEF DESCRIPTION OF THE DRAWINGS

FIG. 1 is a schematic diagram of connection of an H-bridge circuit and a load of an existing electro-stimulation therapeutic instrument for neuromodulation.

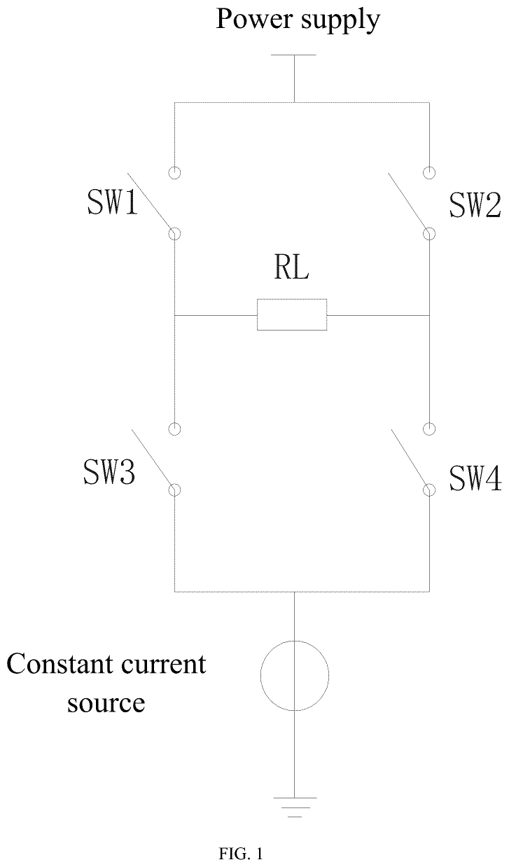

FIG. 2 is a schematic diagram of a closing sequence of a first switch, a second switch, a third switch and a fourth switch and a current flow direction when a forward current is output to a load in a control circuit for an electro-stimulation therapeutic instrument for neuromodulation according to the present disclosure.

FIG. 3 is a schematic diagram of a closing sequence of a first switch, a second switch, a third switch and a fourth switch and a current flow direction when a reverse current is output to a load in a control circuit for an electro-stimulation therapeutic instrument for neuromodulation according to the present disclosure.

FIG. 4 is a module diagram of a control circuit for an electro-stimulation therapeutic instrument for neuromodulation according to the present disclosure.

FIG. 5 is a schematic diagram of connection of a first switch, a second switch, a third switch and a fourth switch and a constant current source when the first switch, the second switch, the third switch and the fourth switch are metal oxide semiconductor (MOS) transistors in FIG. 4.

### DETAILED DESCRIPTION OF THE EMBODIMENTS

The present disclosure will be further described below in combination with the accompanying drawings and the particular embodiments, and it should be noted that on the premise of no conflict, the examples or the technical features described below can be combined freely to form a new example.

The present disclosure solves the problem that a large overshoot current is generated at the moment of switching in the prior art, thereby damaging an electrode plate or causing a tingling sensation to human skin by improving an existing electro-stimulation therapeutic instrument for neuromodulation.

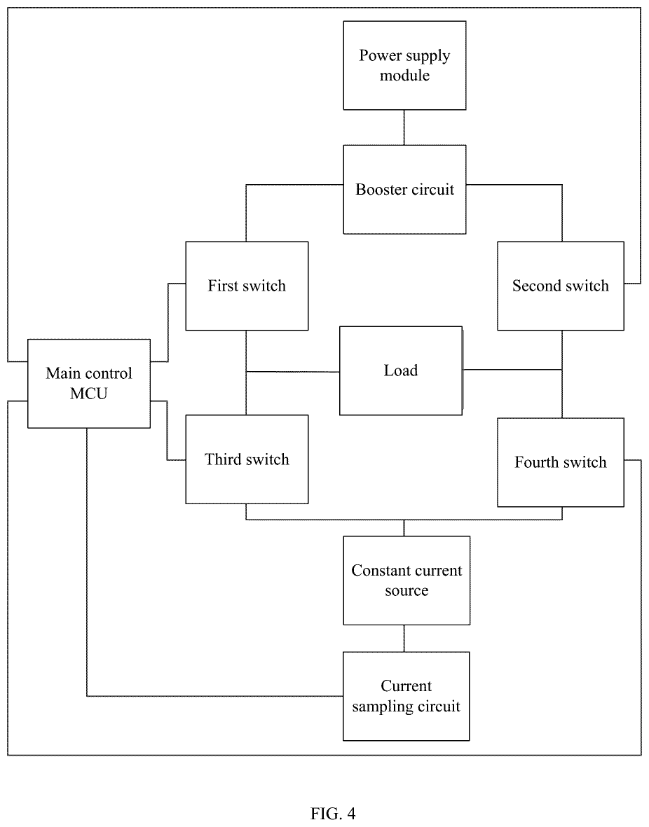

Preferably, the present disclosure provides a control circuit for an electro-stimulation therapeutic instrument for neuromodulation. As shown in FIG. 4, the control circuit includes a first switch SW1, a second switch SW2, a third switch SW3, a fourth switch SW4, a constant current source, a first signal end and a second signal end.

The first switch SW1, the second switch SW2, the third switch SW3, and the fourth switch SW4 are configured as an H-bridge circuit. Specifically, a first end of the first switch SW1 is connected to a power supply, and a second end of the first switch SW1 is electrically connected to the first signal end.

A first end of the second switch SW2 is connected to the power supply, and a second end of the second switch SW2 is electrically connected to the second signal end.

A first end of the third switch SW3 is electrically connected to the first signal end, and a second end of the third switch SW3 is electrically connected to the constant current source.

A first end of the fourth switch SW4 is electrically connected to the second signal end, and a second end of the fourth switch SW4 is electrically connected to the constant current source.

The first signal end is electrically connected to a first end of a load RL, and the second signal end is electrically connected to a second end of the load RL.

The load RL in the example refers to a human body load. Certainly, the load RL may also be considered as an electrode plate and a human body load according to actual conditions. Because, in an actual use process, generally speaking, an output end of the electro-stimulation therapeutic instrument for neuromodulation outputs a current signal to stimulate a human body portion by means of a current. However, in order to ensure the safety of a human body, the purpose of stimulating the human body portion is achieved generally by means of contact of an electrode plate and the human body portion. Therefore, the first signal end and the second signal end may be considered to make contact with the human body portion by means of the electrode plate. That is, the load RL in the example may be considered to be the human body portion and the electrode plate, or the electrode plate. The present application is only intended to explain the connection relation between the control circuit and the load RL.

When a forward current needs to be output to the load RL,
the first switch SWI is controlled to be closed, and the second switch SW2 is controlled to be closed. Since both the first switch SW1 and the second switch SW2 are closed, potentials at two ends of the load RL are equal, and no current flows through the load RL, thereby eliminating initial current surge.

Then, the first switch SW1 is kept closed, the second switch SW2 is kept closed, and the fourth switch SW4 is controlled to be closed. By closing the fourth switch SW4, the current may flow to the constant current source to initialize the constant current source, and the current of the constant current source enters a stable state in advance. In this case, no current flows through the load RL, and no current pulse is generated on the load RL.

Finally, the fourth switch SW4 is kept closed, the first switch SW1 is kept closed, and the second switch SW2 is controlled to be disconnected. By disconnecting the second switch SW2, the current of the constant current source is guided to flow into the load RL, and the current is further transmitted to the electrode plate. In this case, because the constant current source is already in a constant current working state and the current is stable, no overshoot current is generated. That is, no damage is caused to the electrode plate or no tingling sensation is caused to human skin.

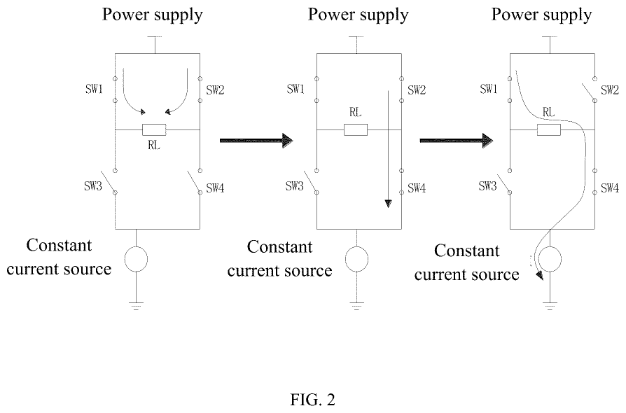

Specifically, as shown in FIG. 2, when 1 in FIG. 2 represents a current trend when a first switch SWI and a second switch SW2 are closed, potentials at two ends of a load RL are equal (that is, potentials at a first signal end and a second signal end are equal), and no current flows through the load RL, so as to eliminate initial current surge.

When 2 in FIG. 2 represents a current trend when a fourth switch SW4 is further closed under the condition that the first switch SWI is closed and the second switch SW2 is closed, the constant current source is initialized, and the current of the constant current source enters a stable state in advance. In this case, no current flows through the load RL, and no current pulse is generated on the load RL.

When 3 in FIG. 2 represents a current trend when the first switch SWI is closed, the fourth switch SW4 is closed, and the second switch SW2 is closed, the current of the constant current source is guided to flow into the load RL and further transmitted to an electrode plate, so as to generate a current pulse. Because the constant current source has entered a constant current working state in advance and does not generate an overshoot current, the current flowing through the load RL has been in a stable state, thereby effectively improving safety, comfort and accuracy of transcutaneous electro-stimulation, and providing better treatment experience and efficiency for a patient.

That is, according to the present disclosure, closing sequences of the switches are controlled to adjust switching time sequences, such that the constant current source enters a normal constant current working mode in advance to ensure that the output current tends to be stable before the current is transmitted to the load RL. Thus, when the current is transmitted to the load RL, the change of the current becomes gentle without sudden change, thereby improving safety and comfort of transcutaneous electro-stimulation, and solving the problem that in the prior art, a large current generated due to current overshoot at the moment when the switch is turned on damages an electrode plate or causes a tingling sensation to human skin.

Similarly, when a reverse current needs to be output to the load RL, the electro-stimulation therapeutic instrument for neuromodulation controls each switch by means of the working modes as follows:
the first switch SWI is controlled to be closed, and the second switch SW2 is controlled to be closed. In this case, since both the first switch SW1 and the second switch SW2 are closed, potentials at two ends of the load RL are equal, and no current flows through the load RL, thereby eliminating initial current surge.

Then, the second switch SW2 is kept closed, the first switch SWI is kept closed, and the third switch SW3 is controlled to be closed. By closing the third switch SW3, the current may flow to the constant current source to initialize the constant current source, and the current of the constant current source enters a stable state in advance. In this case, no current flows through the load RL, and no current pulse is generated on the load RL.

Finally, the third switch SW3 is kept closed, the first switch SWI is controlled to be disconnected, and the second switch SW2 is controlled to be closed. By disconnecting the first switch SW1, the current of the constant current source is guided to flow into the load RL and further is transmitted to the electrode plate. Because the constant current source is already in a constant current working state, no overshoot current is generated. That is, no damage is caused to the electrode plate or no tingling sensation is caused to human skin.

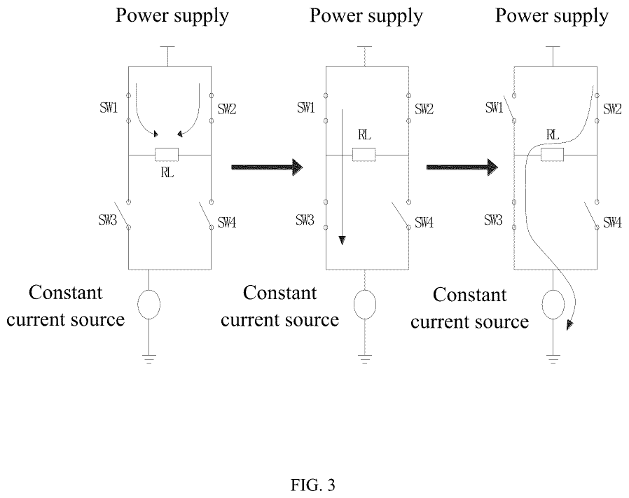

Specifically, as shown in FIG. 3, when 1 in FIG. 3 represents a current trend after a first switch SW1 and a second switch SW2 are closed, potentials at two ends of a load RL are equal, and no current flows through the load RL, so as to eliminate initial current surge.

When 2 in FIG. 3 represents a current trend when a third switch SW3 is further closed under the condition that the first switch SWI is closed and the second switch SW2 is closed, the constant current source is initialized, and the current of the constant current source enters a stable state in advance. In this case, no current flows through the load RL, and no current pulse is generated on the load RL.

When 3 in FIG. 3 represents a current trend when the first switch SWI is disconnected, the third switch SW3 is closed, and the second switch SW2 is closed, the current of the constant current source is guided to flow into the load RL and further transmitted to an electrode plate, so as to generate a current pulse. Because the constant current source has entered a constant current working state in advance and does not generate an overshoot current, the current flowing through the load RL has been in a stable state, thereby effectively improving safety, comfort and accuracy of transcutaneous electro-stimulation, and providing better treatment experience and efficiency for a patient.

Preferably, the electro-stimulation therapeutic instrument for neuromodulation further includes a main control microcontroller unit (MCU). The main control MCU is electrically connected to the constant current source, the first switch SW1, the second switch SW2, the third switch SW3 and the fourth switch SW4, and is configured to control the constant current source and working states of the switches, further control the current flowing through the load RL and control a magnitude of the current flowing through the load RL.

That is, the main control MCU controls closure and disconnection of the first switch SW1, the second switch SW2, the third switch SW3, and the fourth switch SW4, so as to control whether the current of the constant current source flows through the load RL or a direction of the current flowing through the load RL. Preferably, in the present disclosure, the first switch SWI and the second switch SW2 are both positive channel metal oxide semiconductor (PMOS) transistors, and the third switch SW3 and the fourth switch SW4 are both negative channel metal oxide semiconductor (NMOS) transistors.

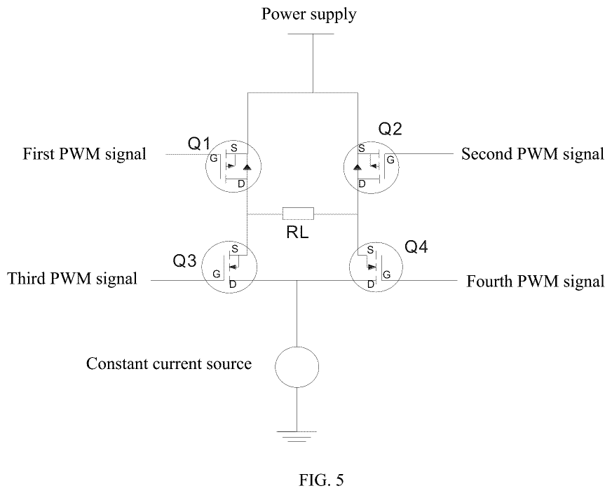

More specifically, as shown in FIG. 5, if the first switch SWI is a first PMOS transistor Q1, the second switch SW2 is a second PMOS transistor Q2, the third switch SW3 is a first NMOS transistor Q3, and the fourth switch SW4 is a second NMOS transistor Q4,
a source of the first PMOS transistor Q1 is connected to the power supply, a drain of the first PMOS transistor Q1 is electrically connected to the first signal end, and a gate of the first PMOS transistor Q1 is connected to a first control signal.

The first control signal refers to a signal configured to control communication and cutoff of the first PMOS transistor Q1, and is provided by the main control MCU.

Similarly, a source of the second PMOS transistor Q2 is connected to the power supply, a drain of the second PMOS transistor Q2 is electrically connected to the second signal end, and a gate of the second PMOS transistor Q2 is connected to a second control signal.

A source of the first NMOS transistor Q3 is electrically connected to the first signal end, a drain of the first NMOS transistor Q3 is electrically connected to the constant current source, and a gate of the first NMOS transistor Q3 is connected to a third control signal.

A source of the second NMOS transistor Q4 is electrically connected to the second signal end, a drain of the second NMOS transistor Q4 is electrically connected to the constant current source, and a gate of the second NMOS transistor Q4 is connected to a fourth control signal.

The main control MCU controls communication and cutoff of the first PMOS transistor Q1, the second PMOS transistor Q2, the first NMOS transistor Q3 and the second NMOS transistor Q4, thereby controlling whether the load RL has pulse output and a pulse direction of the load RL output. Moreover, by controlling the switching time sequences of the MOS transistors, it is ensured that the problem of overshoot current formed at the moment of switch closing is eliminated.

Preferably, the main control MCU is a single chip microcomputer. The first control signal, the second control signal, the third control signal, and the fourth control signal are all pulse width modulation (PWM) signals. That is, the first control signal is a first PWM signal, the second control signal is a second PWM signal, the third control signal is a third PWM signal, and the fourth control signal is a fourth PWM signal. That is, the main control MCU controls closure and disconnection of the MOS transistors by outputting the corresponding PWM signals.

Preferably, the power supply in the present disclosure is a high-voltage power supply. In general, a voltage range of the high-voltage power supply is 50 V to 150 V.

More preferably, the present disclosure further includes a power supply module and a booster circuit. The power supply module is electrically connected to the booster circuit. The booster circuit is configured to boost a power supply provided by the power supply module to form the power supply in the example.

More specifically, the power supply module is a 3.7 V lithium battery. The power supply supplied by the lithium battery may be converted into high-voltage direct current by means of the booster circuit.

Further, the present disclosure further includes a current sampling circuit. One end of the current sampling circuit is electrically connected to the constant current source, and the other end of the current sampling circuit is electrically connected to the main control MCU. The current of the constant current source is collected by means of the current sampling circuit to sent collected current data to the main control MCU, such that the main control MCU determines whether the current of the constant current source is normal according to the collected current data. The main control MCU controls the current of the constant current source by sending a control signal to the constant current source; further collects the actual current of the constant current source by means of the current sampling circuit to determine whether the actual current is consistent with the current controlling the constant current source, so as to determine whether the therapeutic instrument is abnormal; and cuts off the power supply in time and high-voltage stimulation to achieve closed-loop control of nerve stimulation and reduce potential safety hazards when detecting that the therapeutic instrument is abnormal.

Preferably, the current sampling circuit includes a sampling resistor, where the constant current source is electrically connected to the main control MCU by means of the sampling resistor, so as to sample a current value of the constant current source by the main control MCU and monitor the current of the constant current source.

### Example 2

On the basis of Example 1, the present disclosure further provides a control method for an electro-stimulation therapeutic instrument for neuromodulation. The control method is applied to the electro-stimulation therapeutic instrument for neuromodulation used in Example 1.

When a forward current needs to be output to a load RL, the control method includes:
step 11, control a first switch SW1 to be closed, and control a second switch SW2 to be closed.

By simultaneously closing the first switch SW1 and the second switch SW2, initial current surge is eliminated. In this case, potentials at two ends of the load RL are equal, and no current flows through the load.

Step 12, keep the first switch SW1 closed, keep the second switch SW2 closed, and control a fourth switch SW4 to be closed.

By closing the fourth switch SW4, the constant current source is initialized, and the constant current source enters a stable state in advance. In this case, the current flows through the first switch SW1 and the third switch SW3, and then flows into a ground (GND) by means of the constant current source. No current flows through the load RL, and no current pulse is generated on the load RL.

Step 13, keep the fourth switch SW4 closed, keep the first switch SW1 closed, and control the second switch SW2 to be disconnected, so as to make a current of a constant current source flow through the load RL to output the reverse current.

By disconnecting the second switch SW2, the current of the constant current source is guided to be input to the load RL and further is input to an electrode plate connected to the load RL. Because the constant current source has entered a constant current working state, no overshoot current is generated, and the current flowing through the load RL is stabilized.

Similarly, when a reverse current needs to be output to the load, the control method includes:
step 21, control the first switch SW1 to be closed and the second switch SW2 to be closed.

By simultaneously closing the first switch SW1 and the second switch SW2, initial current surge is eliminated. In this case, potentials at two ends of the load RL are equal, and no current flows through the load.

Step 22, keep the first switch SW1 closed, keep the second switch SW2 closed, and control a third switch SW3 to be closed.

By closing the third switch SW3, the constant current source is initialized, and the constant current source enters a stable state in advance. In this case, the current flows through the first switch SW1 and the third switch SW3, and then flows into the GND by means of the constant current source. No current flows through the load RL, and no current pulse is generated on the load RL.

Step 23, keep the third switch SW3 closed, keep the second switch SW2 closed, and control the first switch SW1 to be disconnected, so as to make a current of the constant current source flow through the load RL to output the forward current.

By disconnecting the first switch SW1, the current of the constant current source is guided to be input to the load RL and further is input to the electrode plate connected to the load RL. Because the constant current source has entered a constant current working state, no overshoot current is generated, and the current flowing through the load RL is stabilized.

Switching time sequences of the switches are controlled, such that the current of the constant current source is input to the load RL after the current of the constant current source is stabilized, thereby solving the problem that an electro-stimulation therapeutic instrument in the prior art has an overshoot current at the moment of switching, thereby damaging an electrode plate or causing a tingling sensation to human skin.

The above embodiments are only preferred embodiments of the present disclosure and cannot be used to limit the scope of protection of the present disclosure. Any non-substantial changes or substitutions made by those skilled in the art on the basis of the present disclosure fall within the scope of protection of the present disclosure.

What is claimed is:

1. A control circuit for an electro-stimulation therapeutic instrument for neuromodulation, comprising a first switch, a second switch, a third switch, a fourth switch, a constant current source, a first signal end and a second signal end, wherein a first end of the first switch is connected to a power supply, and a second end of the first switch is electrically connected to the first signal end; a first end of the second switch is connected to the power supply, and a second end of the second switch is electrically connected to the second signal end; a first end of the third switch is electrically connected to the first signal end, and a second end of the third switch is electrically connected to the constant current source; a first end of the fourth switch is electrically connected to the second signal end, and a second end of the fourth switch is electrically connected to the constant current source; and the first signal end is electrically connected to a first end of a load, and the second signal end is electrically connected to a second end of the load;
when a forward current needs to be output to the load, the first switch is controlled to be closed, and the second switch is controlled to be closed; then the first switch is kept closed, the second switch is kept closed, and the fourth switch is controlled to be closed; and finally the fourth switch is kept closed, the first switch is kept closed, and the second switch is controlled to be disconnected, such that a current of the constant current source flows through the load to output the forward current to the load; and
when a reverse current needs to be output to the load, the first switch is controlled to be closed, and the second switch is controlled to be closed; then the first switch is kept closed, the second switch is kept closed, and the third switch is controlled to be closed; and finally, the third switch is kept closed, the second switch is kept closed, and the first switch is controlled to be disconnected, such that a current of the constant current source flows through the load to output the reverse current to the load.

2. The control circuit for an electro-stimulation therapeutic instrument for neuromodulation according to claim 1, further comprising: a main control microcontroller unit (MCU), wherein the main control MCU is electrically connected to the first switch, the second switch, the third switch, the fourth switch and the constant current source; and the main control MCU is configured to control a magnitude of a current flowing through the load from the constant current source.

3. The control circuit for an electro-stimulation therapeutic instrument for neuromodulation according to claim 2, wherein the first switch is a first positive channel metal oxide semiconductor (PMOS) transistor, the second switch is a second PMOS transistor, the third switch is a first negative channel metal oxide semiconductor (NMOS) transistor, and the fourth switch is a second NMOS transistor;
a source of the first PMOS transistor is connected to the power supply, a drain of the first PMOS transistor is electrically connected to the first signal end, and a gate of the first PMOS transistor is connected to a first control signal of the main control MCU; a source of the second PMOS transistor is connected to the power supply, a drain of the second PMOS transistor is electrically connected to the second signal end, and a gate of the second PMOS transistor is connected to a second control signal of the main control MCU; a source of the first NMOS transistor is electrically connected to the first signal end, a drain of the first NMOS transistor is electrically connected to the constant current source, and a gate of the first NMOS transistor is connected to a third control signal of the main control MCU; and a source of the second NMOS transistor is electrically connected to the second signal end, a drain of the second NMOS transistor is electrically connected to the constant current source, and a gate of the second NMOS transistor is connected to a fourth control signal of the main control MCU.

4. The control circuit for an electro-stimulation therapeutic instrument for neuromodulation according to claim 3, wherein the main control MCU is a single chip microcomputer.

5. The control circuit for an electro-stimulation therapeutic instrument for neuromodulation according to claim 4, wherein the first control signal, the second control signal, the third control signal and the fourth control signal are all pulse width modulation (PWM) signals; and
the main control MCU is further configured to adjust duty ratios of the corresponding PWM signals to control closing duration of the corresponding MOS transistors.

6. The control circuit for an electro-stimulation therapeutic instrument for neuromodulation according to claim 2, further comprising: a current sampling circuit, wherein one end of the current sampling circuit is electrically connected to the constant current source, the other end of the current sampling circuit is electrically connected to the main control MCU, and the current sampling circuit is configured to sample an actual current of the constant current source to send the actual current to the main control MCU, such that the main control MCU determines whether the electro-stimulation therapeutic instrument for neuromodulation is abnormal according to the actual current of the constant current source; and the current sampling circuit comprises a sampling resistor, wherein the constant current source is electrically connected to the main control MCU by means of the sampling resistor.

7. The control circuit for an electro-stimulation therapeutic instrument for neuromodulation according to claim 1, wherein the power supply is a high-voltage power supply, and the high-voltage power supply is 50 V to 150 V.

8. The control circuit for an electro-stimulation therapeutic instrument for neuromodulation according to claim 1, further comprising: a power supply module and a booster circuit, wherein the power supply module is electrically connected to the booster circuit; and the booster circuit is configured to boost a power supply supplied by the power supply module to obtain the power supply.

9. The control circuit for an electro-stimulation therapeutic instrument for neuromodulation according to claim 8, wherein the power supply module is a 3.7 V lithium battery.

10. A control method for an electro-stimulation therapeutic instrument for neuromodulation, wherein the control method is applied to the control circuit for an electro-stimulation therapeutic instrument for neuromodulation of claim 1,
when a forward current needs to be output to a load, the control method comprises:
step 21, controlling a first switch to be closed, and controlling a second switch to be closed;
step 22, keeping the first switch closed, keeping the second switch closed, and controlling a fourth switch to be closed; and
step 23, keeping the fourth switch closed, keeping the first switch closed, and controlling the second switch to be disconnected, so as to make a current of a constant current source flow through the load to output the forward current; and
when a reverse current needs to be output to the load, the control method comprises:
step 11, controlling the first switch to be closed and the second switch to be closed;
step 12, keeping the first switch closed, keeping the second switch closed, and controlling a third switch to be closed; and
step 13, finally, keeping the third switch closed, keeping the second switch closed, and controlling the first switch to be disconnected, so as to make a current of the constant current source flow through the load to output the reverse current.
